(19) United States
(12) Patent Application Publication
(10) Pub. No.: US 2010/0042134 A1
(43) Pub. Date: Feb. 18, 2010

(54) Nostril dilator

(76) Inventor: Abraham Wien, Miami, FL (US)

Correspondence Address: WILEY REIN LLP, 1776 K. STREET N.W., WASHINGTON, DC, 20006, US

(21) Appl. No.: 12/148,486

(22) Filed: Apr. 18, 2008

(51) Int. Cl. A61M 29/00

(52) U.S. Cl. 606/199

(57) ABSTRACT

A device for relieving congestion in restricted nasal passages, by the dilation thereof with an internal prosthesis placed within the nasal passages of an individual. This prosthesis comprises an adjustable bow having a pair of appendages extending from each end thereof. A separate tensioning element engages complimentary features on/in the bow to limit flexure thereof, and thereby provide a counterforce to bending of the bow. Accordingly, when the bow is flexed or bent, the extent of flex or bending of the bow is constrained by the tensioning element. The combined effect of the natural recoil of the resilient bow from the distended to flat position, combined with the counter forces imparted to the bow by the tensioning element, causes each appendage on the end of the bow to exert a constant pressure within each nasal passage to maintain such passages in a relatively open position.

### BACKGROUND OF THE INVENTION

1. Field of the Invention

This invention relates to a device. More specifically, this invention is directed to a device for relieving congestion in restricted nasal passages, by the dilation thereof with an internal prosthesis placed within the nasal passages of an individual. This prosthesis is adjustable, and under tension, so as to thereby exert constant pressure within each nasal passage to maintain such passages in a relatively dilated/open position.

2. Description of the Prior Art

The natural tendency is for human beings to breath through their nose, unless otherwise restricted from doing so. When such restriction is encountered, for any one of number of reasons, breathing is shifted to the mouth, which in turn can result in snoring, and possibly other conditions associated therewith (e.g. restrictive apnea).

Over the years a number of devices have been developed to lessen the nasal restriction and improve nasal breathing patterns. These devices fall into two broad categories: internal prosthesis and external prosthesis.

The more widely accepted of these devices, an external prosthesis, comprises a spring-like element that has an adhesive coating on one surface thereof. U.S. Pat. No. 5,769,089 (Hand et al., issued Jun. 23, 1998) is representative of this category of device. In brief, the Hand device comprises an external “nasal splint” of composite construction (3 layers). The Hand device includes a planar sheet, a strip of high density polyethylene and an adhesive layer. In practice, the Hand device is placed, in a conforming relation, on surface of the nose, with the adhesive layer proximate to, and in an adherent contact relationship with the skin. Upon placement and adherence of the Hand device on the nose, the high flexural modulus of the plastic strip recoils, and pulls the nostrils of the wearer outward while the external nasal splint is worn. By pulling the nostrils outward, the external nasal splint reportedly opens the nasal passage, thereby improving breathing and alleviating problems such as snoring.

The internal nasal prosthesis are represented by U.S. Pat. No. 6,386,197 (to Miller, issued May 14, 2002); U.S. Pat. Nos. 4,201,237 6,238,411 (to Thorner, issued May 29, 2001); and U.S. Patent (to Slater, issued May 6, 1980).

The Miller device includes a pair of conical shaped tubular element that is placed within each nostril. These tubular elements have what can best be described a radial array of “hose barbs” to prevent their being dislodged during use. Presumably the main advantage of the Miller design is aesthetic—the tubular elements are not visible during use.

The Thorner prosthetic device comprises an “internal nasal dilator” of composite construction. More specifically, the Thorner internal dilator is the product of a combination of several elements; specifically a V-shaped member, such as a flat spring is provided, with resilient pad means attached at each end thereof. The V-shaped member or the flexible nasal strip is then attached to the nose by the wearer at the central portion (outside bottom), by means of an adhesive element which extends from or near the free unattached ends of strip. The V-shaped spring element of the device, with the pad means, is inserted into each nostrils of the wearer, so the spring can spread the outer walls of the nose, and thereby increase the nasal opening for improved breathing. The adhesive portions at the free strip-ends are pressed against the outer surfaces of the nose by the wearer for retaining the pad means in place, inside the nose. The bias on the spring element is pre-set and does not appear to adjustable to accommodate different sized nostril openings.

The Slater prosthetic device comprises a “nasal dilator” in the form of “U” shaped bow. The “U” shaped bow has two “legs”, one of which is placed in each nostril. Because of the tension on the bow, and the outward pressure exerted by each leg of the bow on each nostril, the nostrils are dilated. In order to maintain the requisite degree of tension/pressure on each nostril by the bow, an “expander” or “bridge” is placed between each leg of the bow to insure a contact spacing (spreading) of the legs of the bow. The “expander” or “bridge” is manually adjusted, to lock the legs of the bow in a pre-set configuration, prior to the insertion of the bow into the nostrils.

As is evident from the foregoing, neither of the design configurations of the presently available internal or external prosthetic devices for relieving nasal restriction, are without their shortcomings. More specifically, the external devices, of the type illustrated by the Hand patent, are limited in the amount of expansive pressure that can be exerted upon each nostril by the adhesive forces that can be applied to the external surface of the nose. Similarly, the internal devices of the type illustrated by the Thorner and Slater patents lack the ability to apply a constant pressure from with each nostril without periodic manual adjustment and the repositioning such device on a periodic basis. Accordingly, while the external device prosthetic device of Hand is favored because of the constant pressure, spring tensioning element incorporated therein, it lacks the ability for user modulation; and, is otherwise dependent upon adhesives for its contact with the nasal passages. Accordingly, there continues to exist a need for improvement of the nasal prosthesis for relief of nasal congestion. Ideally such improvement should incorporate the advantages of the external “nasal splint” of Hand, and the internal nostril expander of Slater.

### OBJECTS OF THE INVENTION

It is the object of this invention to remedy the above as well as related deficiencies in the prior art.

More specifically, it is the principle object of this invention to provide an internal nasal prosthesis for placement within the nostrils of an individual to enhance breathing through the nose.

It is yet another object of this invention, to provide an internal nasal prosthesis for placement within the nostrils of an individual to enhance breathing through the nose, by application of constant pressure within each nasal passage, so as to maintain each nostril in a relative open position.

It is still yet another object of this invention, to provide an internal nasal prosthesis for placement within the nostrils of an individual wherein the prosthesis includes means for adjusting the relative constant pressure of the prosthesis on the nostrils, so as to maintain each nostril in a relative open position, notwithstanding changes in facial expression or sleep state.

Additional objects of this invention include a method for the treatment of nasal congestion and related breathing disorders with a mechanical prosthesis.

### SUMMARY OF THE INVENTION

The above and related objects are achieved by providing an internal nasal prosthesis having three basic functional components:
a resilient flexible bow having a medial section and an appendage on each end thereof, wherein such bow comprises a planar strip of plastic material, having an appendage extending from each end thereof;
a pair of pads, or cushioning elements, on each end of the appendages, for engagement of the surface of the internal nasal passages of an individual; and,
a tensioning element or spreader, for engagement with the resilient flexible bow, to cause the flexible appendages of the bow to engage and exert constant pressure on each nostril, and thereby relieve breathing difficulties.

In the preferred embodiments of this invention, the internal nasal prosthesis of this invention includes a bow have a series of détentes and/or apertures associated therewith, for attachment of the tensioning element/spreader at different positions on the bow, so as to pre-calibrate the tension on the bow, prior to its insertion in the nostrils of an individual.

In one of the preferred embodiments of the invention, the tensioning element comprises an elastic strap having a pair of surface modifications (posts) for engagement with détentes and/or apertures along the length of the bow. Conversely, the medial section of the bow can include a series of posts, and the elastic strap a corresponding series of holes, for engaging the posts on the medial section of the bow. Thus, the tensioning element can be repositioned, as desired, to achieve the requisite degree of tension on the appendages, without consequent discomfort from excessive pressure on the tissues of the nostrils.

In another of the preferred embodiments of the invention, the internal nasal prosthesis of this invention includes a leaf spring-like element that can be wedged between each of the opposing legs of the bow, so as to force these legs apart, and thereby cause the appendages at the end of the bow to exert a relatively constant pressure on the nostrils and thereby relief from nasal congestion.

In another of the preferred embodiments of the invention, the cushioning pads on the ends of the appendages of the nasal prosthesis can be pretreated with a nasal decongestant, or the user can apply a decongestant to the pads prior to the placement of the internal nasal prosthesis in his nose.

### BRIEF DESCRIPTION OF THE DRAWINGS

FIG. 1 depicts a perspective view of an internal nasal prosthesis of this invention.

FIG. 2 depicts an enlarged and exploded view of the internal nasal prosthesis of FIG. 1 at Section AA, wherein an elastic tensioning element is positioned on the underside of the medial section of the bow.

FIG. 3 depicts an internal nasal prosthesis of this invention prior to its installation in the nostrils of an individual.

FIG. 4 depicts an alternative configuration of an internal nasal prosthesis of this invention, wherein the tensioning element, in the form of a leaf spring-like member, is position proximate the top-side of the medial section of the bow.

FIG. 5 depicts an alternative configuration of this invention wherein the internal nasal prosthesis in integrated with a canula of an auxiliary oxygen supply system

FIG. 6 is an alternative embodiment of the device of FIG. 1.

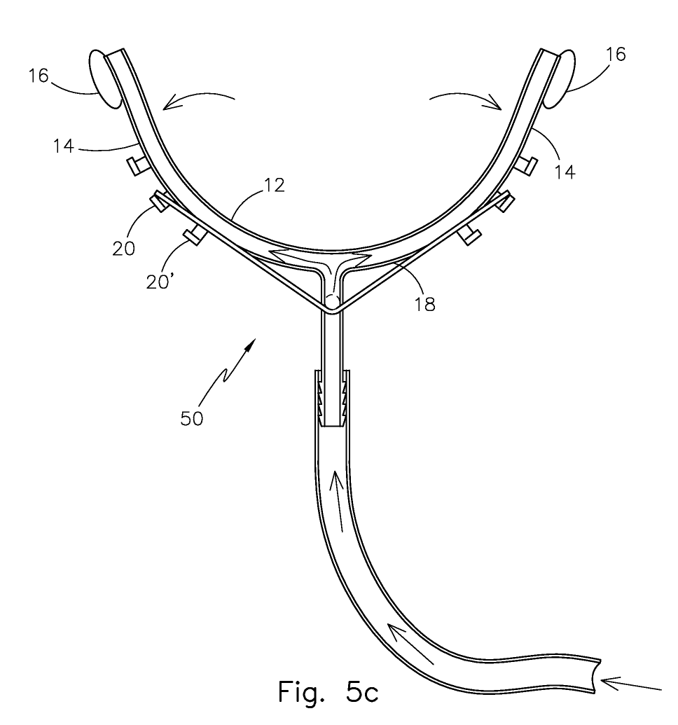

FIG. 7 is an enlarged and exploded view of the internal nasal prosthesis of FIG. 6 at Section BB.

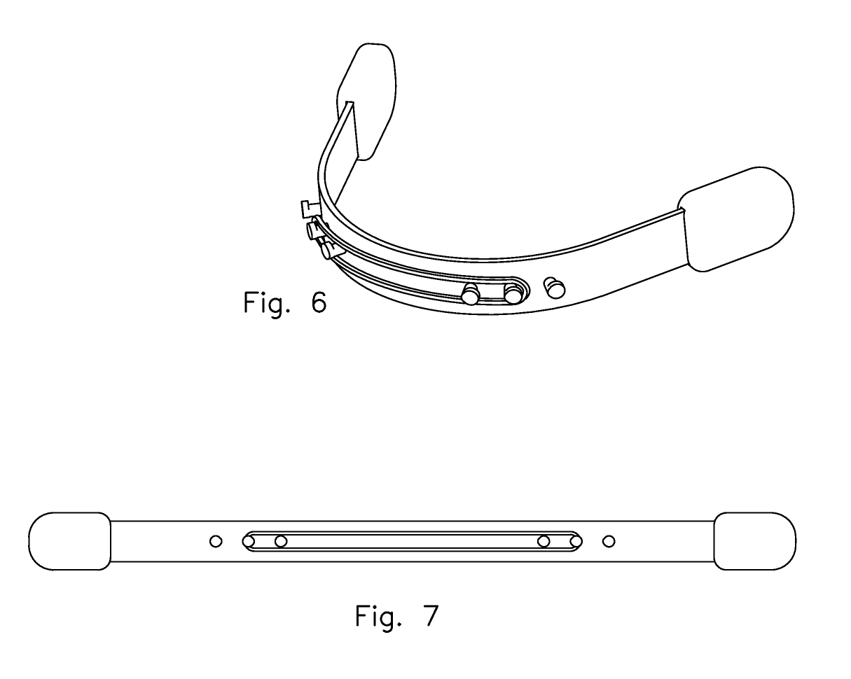

### DESCRIPTION OF THE INVENTION INCLUDING PREFERRED EMBODIMENTS

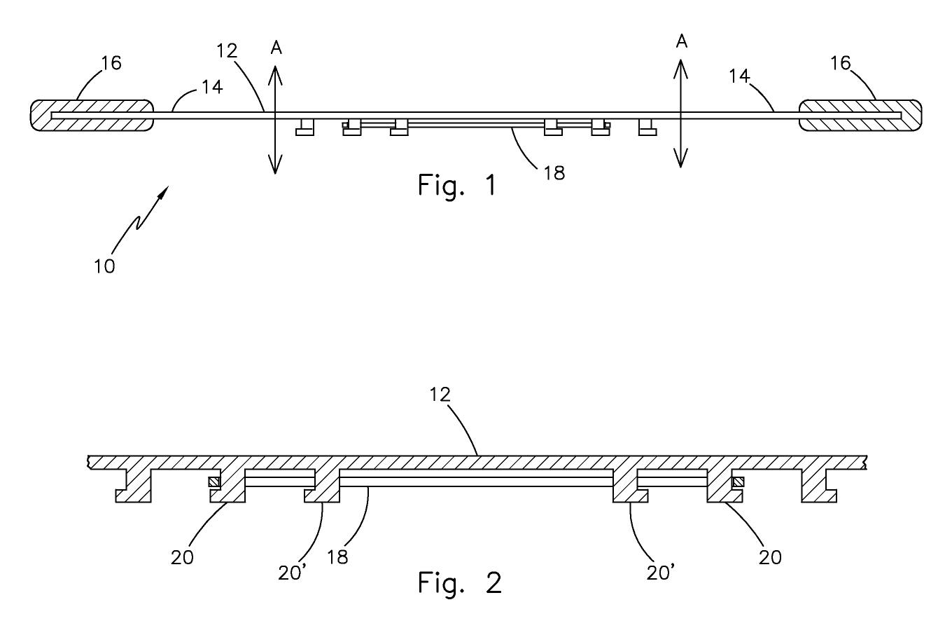

The preferred embodiment of the internal nasal prosthesis (10) of this invention is depicted in FIG. 1. The basic configuration of the prosthesis has three, and preferable four, basic elements:
a resilient flexible bow (12) which preferably comprises a planar strip of plastic material capable of bending and taking the shape of a “V” or “U”.
an appendage (14) on each end of the bow, preferably comprising a tubular member, of an internal diameter sufficient to be fitted on each end of the bow;
a pair of pads (16), or cushioning elements, on the free end of each of the appendages, for engagement of the surface of the internal nasal passages of an individual; and,
a tensioning element or spreader (18, 18′), for engagement with one of more surface features, (e.g. détentes, holes and/or posts), so as to provide for incremental positioning (20, 20″) the tensioning element relative to the surface of the resilient flexible bow (12).

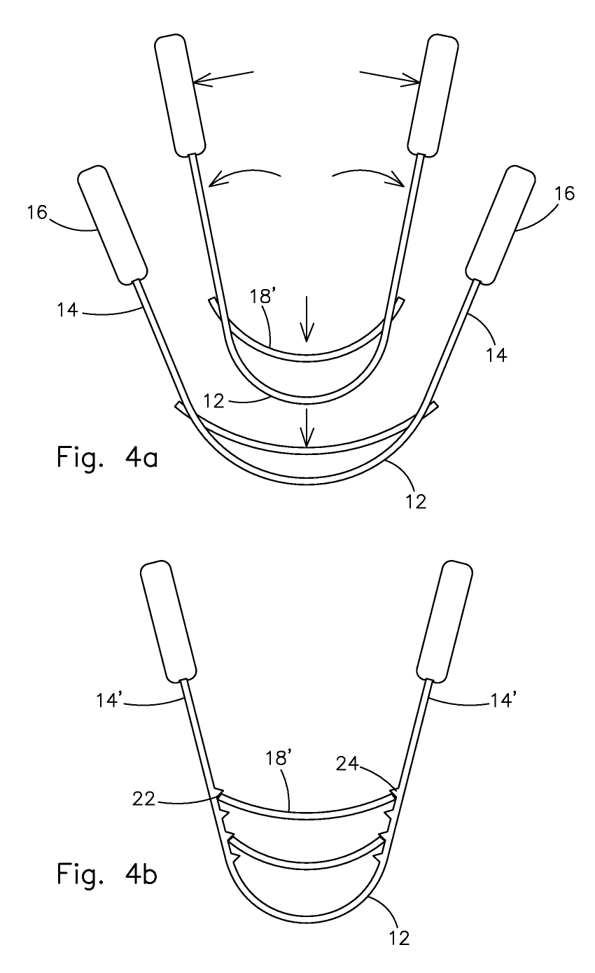

In practice, the bow (12) of the nasal prosthesis (10) is initially bent or flexed to form a “U” or “V”, (as shown in FIG. 3), and one appendage (14) on each end thereof insert into the right and left nostril, respectively. In the embodiment of the invention illustrated in FIGS. 1 & 2, the bow and the appendages are structurally distinct from one another. This does, however, not imply that these two functional components cannot be combined in a single unitary structure or component; and, the fact that they are depicted as separate and distinct in FIGS. 1 & 2, does not exclude a unitary alternative.

Because of the resilient nature of the bow (12), it has a degree of inherent resistance to bending, and will recoil to a degree, so as to cause the appendages (14) on the ends thereof to spread out engage each nostril. Notwithstanding, the amount of pressure created by such recoil, the pressure exerted by the recoil of the bow alone is generally insufficient to substantially dilate the nostrils, and to effect the desired decongestive effect. In the prosthesis depicted in FIGS. 1 & 2, a tensioning element (18) on the underside of the bow (10), increases the resistance of the bow to deformation (bending), by exertion of a constant opposing force on the underside of the bow.

The tensioning element (18) is initially pre-position on the bow at a given location, and it may be moved or adjusted, depending upon the desired level of pressure to be asserted within each nostril, and/or to relieve any discomfort caused by pressure within each nostril caused by the prosthesis.

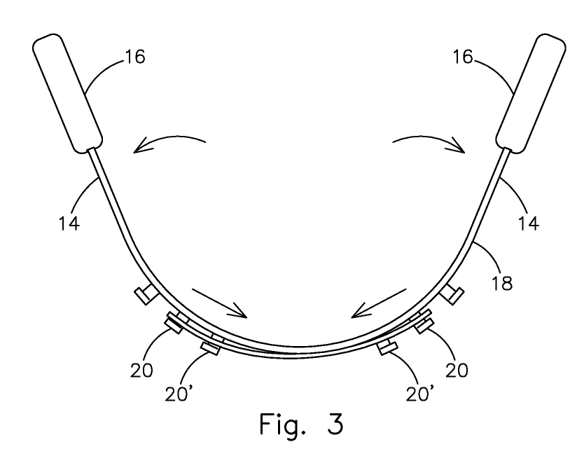

FIG. 2 depicts an enlarged view of the tensioning element of the prosthesis of FIG. 1. This tensioning element is an elastic or rubber strap having a series of projections, (e.g. posts, pegs etc.), arranged along its length. In FIG. 2, the tensioning strap has a pair of projections positioned toward either end of the tensioning strap. The relative spacing of these projections corresponds to the spacing of a series holes along the medial section of the bow. Thus, in FIGS. 1 & 2, the tensioning strap engages the bow by insertion of these projections, on the rubber strap, into the corresponding holes in the bow. Where increased tension on the bow is desired, the tensioning strap is reposition by removing the elastic/rubber strap from the bow, and reinsertion of the projections thereof into a new (more outboard) pair of holes. This process is repeated until the tension exerted by the tensioning strap on the bow is sufficient for the prosthesis to enhance the ease of breathing of an individual; and, yet insufficient to cause to discomfort to the individual when placed with his nostrils. It is noted that the corresponding complimentary elements on each the tensioning strap and the medial section of the bow can be reversed, with the projections or pegs being formed on the surface of the medial section of the bow, and corresponding holes molded or formed in the elastic strap.

FIG. 3 depicts the prosthesis of this invention in a flexed position, prior to the insertion into the nostrils of an individual. In this configuration, the prosthesis is bent into the shape of a “U” shaped device, to facilitate placement of the appendages thereof into each nostril of an individual. The arrows in the illustration depict the forces on the bow, specifically the “pull” of the tensioning strap on the underside of the bow; and, the consequent direction of the spreading action, (force or pressure) on the pads. As noted above, the tensioning strap can be re-positioned, as appropriate to increase or decrease the “pull” on the underside of the bow, and thereby change the spreading force on the pads of the appendages.

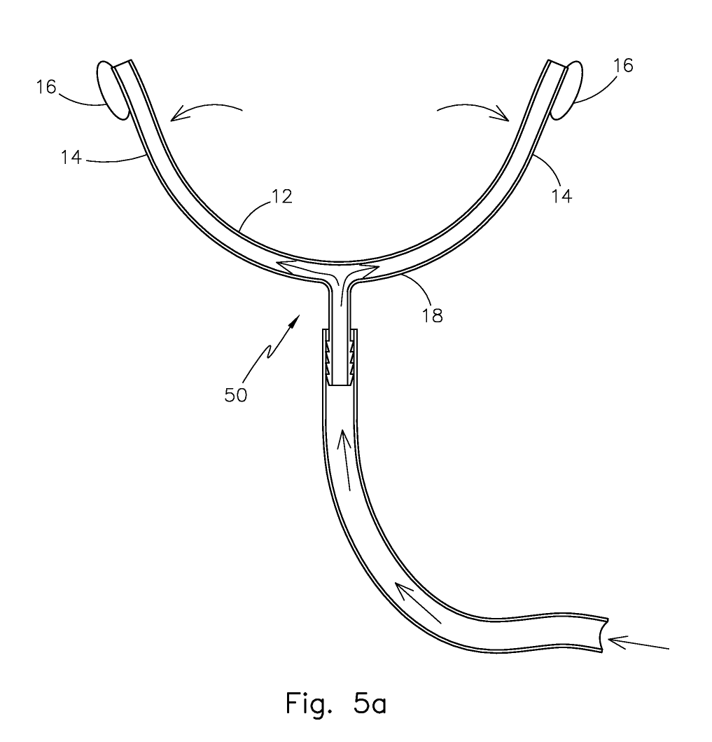

FIG. 4 depicts an alternative configuration of the prosthesis on this invention. In this alternative embodiment, the elastic tensioning element of FIGS. 1-3, is replaced with a leaf spring-like member (18′), which is placed between the appendages of the bow, so as to engage a series of détentes on the surface of the bow. In FIG. 4, this leaf spring-like member comprises a planar resilient plastic strip, having a concave bend corresponding to the curvature of the prosthesis depicted in FIG. 3

This leaf spring-like member (18′) is also provided with a series of surface features (22) which are complimentary of a series of surface features (24) on the interior surface of the bow (12). Thus, when the leaf spring (18′) is placed within the space between the appendages (14′) and engages the surface features (24) on the surface of the bow (12), an outward or expanding pressure is exerted upon the bow. This outward or expanding pressure translates a constant pressure to the appendages (14′), and thereby reduces nasal restriction and enhances breathing.

Alternatively, tensioning means can be engineered into the medial section of the bow itself by simply constructing the bow from two separate components that are configurable to form a medial section of varying length. For example, in this alternative, the medial section of the bow is assembled from two tubular components that each telescope relative to one another. Similarly, each of the foregoing two tubular elements of the medial section of the bow can include a threaded end wherein each threaded component can be screwed into the other, or into an intermediary coupler, in the form of a collar. The shorter the resulting bow, the more resistance to bending and, thus, the greater pressure that can be exerted by the pads of the appendages on the nostrils.

The pads/cushioning elements on the ends of the appendage (14. 14′) can comprise a cotton-like material (e.g. Q-Tip® or foam-like element). In one of the preferred embodiments of this invention, these pads can be pretreated with a nasal decongestant, or other medicinal agent, to help relieve breathing discomfort. Alternatively, the individual user may choose to apply his own decongestant of choice to these pads prior to the placement of the prosthesis in this his nostrils.

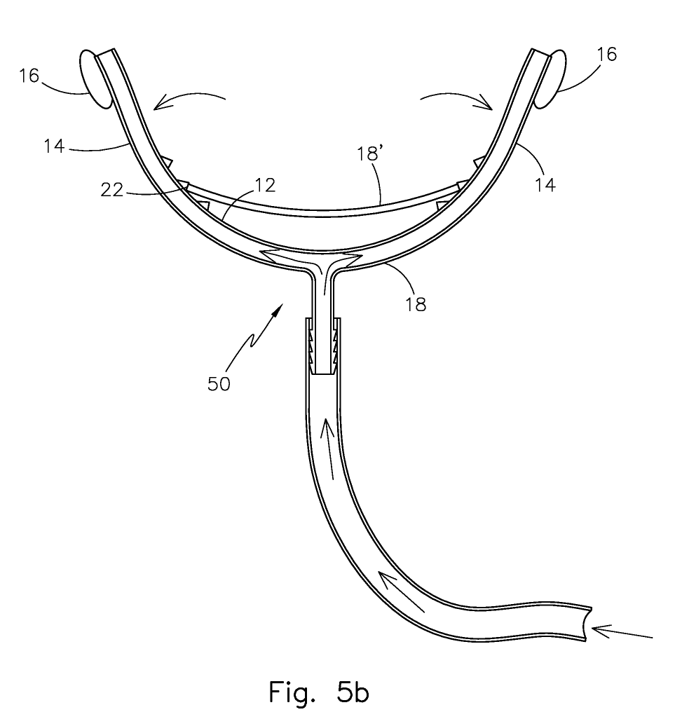

FIG. 5 depicts the nasal prosthesis of this invention as an integral component of canula of an auxiliary oxygen supply system. In this embodiment of this invention a canula (50) is fitted on each lateral end thereof with an appendage (14). The tensioning means for the appendages can be either the tensioning means or leaf-spring like element referenced hereinabove in the description of FIGS. 1-4.

The basic configuration of the prosthesis of this invention can be further modified by addition or alteration of one of more of its basic components (e.g. use or provision of replaceable pad or appendages), without departure from the scope of the invention, which is defined in the following set of claims.

What is claimed is:

1. An internal nasal prosthesis for relief of breathing congestion, comprising:
A. A resilient flexible bow having a medial section, and an appendage on each end thereof;
B. A pair of pads, or cushioning elements, on each end of said appendages, for engagement of the surface of the internal nasal passages of a nose of an individual; and,
C. A tensioning element adapted for engagement with said medial section of said resilient flexible bow, said tensioning element providing a counter force to bending of said flexible bow, so as to cause said appendages of said bow, when placed within a nose of an individual to engage and exert constant pressure on each nostril, and thereby relieve breathing difficulties.

2. The internal nasal prosthesis of claim 1, wherein said bow comprises a medial section, including a planar strip of plastic material, and tubular appendage extending from each end of said medial section of said bow.

3. The internal nasal prosthesis of claim 1, wherein said appendage on each end of thereof, includes a pad or cushion for pressure contact with each nostril of an individual's nose.

4. The internal nasal prosthesis of claim 1, wherein said tensioning element includes means associated therewith for engagement with complimentary means on said medial section of said resilient flexible bow, so at to, upon engagement with said bow, increase resistance of said bow to bending.

5. The internal nasal prosthesis of claim 4, wherein said tensioning element is selected from the group consisting of: (a) an elastic strap having a series of projections thereon, for engagement with a series of corresponding holes in said medial section of said bow; (b) an elastic strap having a series of holes formed therein, for engagement with a series of corresponding posts formed along the surface of said medial section of said bow; and any combination of (a) and (b).

6. The internal nasal prosthesis of claim 4, wherein said tensioning element comprises a resilient leaf spring-like member having a series of projections at near each end thereof for engagement with a series of corresponding détentes on the surface of said medial section of said bow, so as to cause the appendages of said bow to spread apart, and thereby increase resistance of said bow to bending.

7. The internal nasal prosthesis of claim 1, wherein said pad/cushioning elements on the ends of said appendages are pre-treated with a medicinal agent prior to placement in an individual's nostrils

8. The internal nasal prosthesis of claim 4, wherein tensioning element of said prosthesis includes both an elastic strap and a leaf spring-like member of engaging said bow, to increase resistance of said bow to bending.

9. The internal nasal prosthesis of claim 4, wherein said medial section of said bow comprises two elements, a first element and a second element, each of said first and said second element including means for interlocking said first element with said second element, so as to form a unitary structure of differing structural characteristics with different resistance to deformation or bending.

10. The internal nasal prosthesis of claim 4, wherein said medial section of said bow comprises two elements, a first element and a second element, each of said first element and said second element engaging one another at different positions to form a bow of variable length.
